(19) United States
(12) Patent Application Publication
(10) Pub. No.: US 2011/0004288 A1
(43) Pub. Date: Jan. 6, 2011

(54) INTRAVASCULAR IMPLANTABLE DEVICE HAVING INTEGRATED ANCHOR MECHANISM

(76) Inventor: Terrance Ransbury, Chapel Hill, NC (US)

Correspondence Address: PATTERSON THUENTE CHRISTENSEN PEDERSEN, P.A., 4800 IDS CENTER, 80 SOUTH 8TH STREET, MINNEAPOLIS, MN, 55402-2100, US

(21) Appl. No.: 12/815,355

(22) Filed: Jun. 14, 2010

Related U.S. Application Data
(60) Provisional application No. 61/186,805, filed on Jun. 12, 2009.

(51) Int. Cl. A61F 2/82; A61F 2/84

(52) U.S. Cl. 623/1.15; 623/1.23; 623/1.36

(57) ABSTRACT

Devices, systems and methods are provided for an intravascular implantable device having an integrated anchor mechanism that can be deployed by compressing the anchor in an axial direction. Various embodiments address the various issues presented by the prior art and/or improve upon the prior art devices, methods and systems for anchoring an intravascular implantable device within a vessel.

### RELATED APPLICATIONS

This application claims priority to U.S. Provisional Patent Application No. 61/186,805, filed Jun. 12, 2009, which is hereby incorporated by reference. The present application is related to, but does not claim the benefit of, U.S. Pat. No. 7,617,007 and U.S. Published Application Nos. 2007/0265673, 2008/0147168, 2008/0154327, 2008/0167702, 2009/0163927, and 2009/0192579, the disclosures of which are hereby incorporated by reference.

### FIELD OF THE INVENTION

The present invention relates generally to surgical devices and methods for retaining medical devices within the body, and more specifically to devices, systems and methods for anchoring an intravascular implantable device within a vessel.

### BACKGROUND OF THE INVENTION

Implantable medical devices such as pacemakers, defibrillators, and implantable cardioverter defibrillators (“ICDs”) have been successfully implanted in patients for years for treatment of heart rhythm conditions. Pacemakers are implanted to detect periods of bradycardia and deliver low energy electrical stimuli to increase the heart rate. ICDs are implanted in patients to cardiovert or defibrillate the heart by delivering high energy electrical stimuli to slow or reset the heart rate in the event a ventricular tachycardia (VT) or ventricular fibrillation (VF) is detected. Another type of implantable device detects an atrial fibrillation (AF) episode and delivers electrical stimuli to the atria to restore electrical coordination between the upper and lower chambers of the heart. Still another type of implantable device stores and delivers drug and/or gene therapies to treat a variety of conditions, including cardiac arrhythmias. The current generation for all of these implantable devices are typically can-shaped devices implanted under the skin that deliver therapy via leads that are implanted in the heart via the patient's vascular system.

Next generation implantable medical devices may take the form of elongated intravascular devices that are implanted within the patient's vascular system, instead of under the skin. Examples of these intravascular implantable devices are described, for example, in U.S. Pat. Nos. 7,082,336, 7,529,589 and 7,617,007, and U.S. Published Patent Application Nos. 2004/0249431 and 2008/0167702. These devices contain electric circuitry and/or electronic components that are hermetically sealed to prevent damage to the electronic components and the release of contaminants into the bloodstream. Due to the length of these intravascular implantable devices, which in some cases can be approximately 10-60 cm in length, the devices generally are designed to be flexible enough to move through the vasculature while being sufficiently rigid to protect the internal components.

Securing such an implantable device within the vasculature is one of the challenges for this next generation of intravascular implantable devices. In addition to the mechanical and operational considerations related to an anchoring system, there are physiological and biological implications for the patient, as well as considerations for how an anchoring system may affect the manner in which the implantable device delivers therapy.

An early approach to anchoring intravascular implantable devices was to secure the body of the device within the inferior or superior vena cava of the patient, such as described in U.S. Pat. No. 7,082,336 and U.S. Published Patent Application No. 2004/0249431. In some embodiments, the anchoring system could be integrated with the body of the intravascular implantable device so as to comprise an asymmetrically expandable anchor. In other embodiments, the anchor was provided separately from the device and was used to pin (or “sandwich”) the body of the intravascular implantable device in position between the anchor and the vessel wall. In still other embodiments, a lead extending from a distal end of the body of the intravascular device would also be anchored in the vasculature, such as in a subclavian vein. In some embodiments, a delivery sheath was employed to maintain the anchor in a compressed position until it was desired to be deployed. In some embodiments, a vessel liner was deployed prior to delivery and anchoring of the device, such that the device was pinned against the liner by the stent.

An alternative integrated anchoring system for an intravascular implantable device is described in some of the embodiments shown in U.S. Published Patent Application No. 2005/0228471. This alternative integrated anchoring system utilized a radially expandable member positioned proximate the middle of the body of the device to secure the device. In some embodiments, the radially expandable member generally centered the device body coaxially within the diameter of the vessel. In other embodiments, two or more radially expandable members were used to secure the middle of the body of the device within a vessel. The anchor arrangements disclosed herein are secured to the device such that the device is carried within an interior portion of the anchor, in contrast to other anchor arrangements wherein the device is sandwiched between the anchor and a vessel. Additional anchoring embodiments similar to those described above are also described in U.S. Pat. No. 7,529,589.

As described in U.S. Published Patent Application Nos. 2008/0147168 and 2008/0167702, anchoring the intravascular implantable device within a vessel superior to the heart may be advantageous to prior anchoring locations. In one embodiment, the implantable device may include a tether portion coupled to or integrated with a distal portion of the device, wherein the tether portion includes or is coupleable with a cleat configured to mechanically engage a stent anchor within a vessel. In such an arrangement, the tether portion is secured to the anchor proximate the vessel wall.

A further anchoring arrangement is described in U.S. Pat. No. 7,519,424, wherein the implantable device is retained within the vasculature above the atrium in one embodiment by a tether portion extending from the device body. The tether includes an anchoring member, and the tether is configured to extend through a vascular wall to a suitable fixation site such as directly to the external wall of the vessel, or tissue external to the vessel.

While intravascular implantable devices represent a significant improvement over conventional implantable devices that are implanted subcutaneously, there are opportunities to improve and refine the designs for such intravascular devices. Accordingly, it would be desirable to provide for an improved design of an anchoring arrangement for an intravascular implantable device.

### SUMMARY OF THE INVENTION

In accordance with embodiments of the present invention, devices, systems and methods are provided for an intravascular implantable device having an integrated anchor mechanism that can be deployed by compressing the anchor in an axial direction. Various embodiments address the various issues presented by the prior art and/or improve upon the prior art devices, methods and systems for anchoring an intravascular implantable device within a vessel.

In one embodiment, a method of implanting an intravascular implantable device into a vasculature of a patient includes creating an access to a vasculature of a patient, advancing the intravascular implantable device until it is fully within the vasculature of the patient, the intravascular implantable device including an integrated anchor and an elongated enclosure containing a battery and electronic components, and deploying the anchor by compressing the anchor in an axial direction, so as to secure the intravascular implantable device within the vasculature of the patient.

In another embodiment, an intravascular implantable device includes an elongated enclosure containing a battery and electronic components, and an integrated anchor mechanism operably coupled to the elongated enclosure, the anchor mechanism configured to be deployable in response to compression of the anchor mechanism in an axial direction.

In another embodiment, a system includes an intravascular implantable device, an anchor having a longitudinal axis and a plurality of leg members, a pullwire configured to releasably couple to the anchor, and an anchor delivery catheter. Each leg member includes a plurality of joints. A proximal portion of the pullwire is received by the anchor delivery catheter and the anchor delivery catheter is configured to retract the pullwire with respect to the anchor delivery catheter such that the anchor is compressed along the longitudinal axis and deployed so as to secure the intravascular implantable device within the vasculature of a patient.

In another embodiment, a method includes providing an intravascular implantable device having an integrated anchor mechanism, the intravascular implantable device including an integrated anchor and an elongated enclosure containing a battery and electronic components, and providing instructions for implanting the intravascular implantable device into a vasculature of a patient. The instructions include creating an access to a vasculature of a patient, advancing the intravascular implantable device until it is fully within the vasculature of the patient, and deploying the anchor by compressing the anchor in an axial direction so as to secure the intravascular implantable device within the vasculature of the patient.

Embodiments of the invention offer improvements on the prior approaches to anchoring an implantable device within the vasculature of a patient. For example, in prior approaches where the anchor is provided separate from the device and is configured to sandwich the device (or a portion of the device) against a vessel wall, a potential exists for slippage or pull-out of the device from the anchor, or dislodgement of the anchor from the vessel wall. Further, proper alignment of the anchor with respect to the device can be critical. Anchoring outside a desired zone or range within which the anchor should be deployed may result in incomplete or ineffective anchoring of the device within the vessel.

The prior approaches to anchoring an implantable device within the vasculature of a patient typically have relied on radially self-expanding anchor structures. In order to be delivered through the vasculature to the anchor location, such self-expanding anchors must be retained in a radially compressed position. For non-integrated anchor embodiments, the use of a specialized anchor delivery tool can assist with accurate, repeatable anchor deployment. Such tools cannot be used for anchors integrated with an intravascular implantable device, nor can the complex implantable device be easily fitted with features similar to those found on anchor delivery catheters. The inclusion of additional tools and devices near the anchoring area also increases the likelihood of accidental capture of a tool by the anchor when the anchor is released from the delivery tool. Additionally, in the event an intravascularly implanted device must be removed from the patient, difficulties may exist with prior radially deployed anchoring approaches.

Due to variations in vessel diameters among the patient population, it is unlikely that a single sized, radially deployed anchor will perform satisfactorily for all patients. Typical self-expanding radial anchors are designed to be used within a narrow predetermined range of expanded diameters. A patient will either undergo an MRI prior to the implant to determine the vessel size, or a vascular measurement is taken during the device implant. Thus, hospitals must maintain an inventory of anchors of multiple sizes and/or configurations, increasing the cost of the procedure.

Disclosed and described herein are anchoring embodiments that reduce the likelihood of slippage or pull-out of an IID from an anchor or of the anchor from the vessel, are adaptable to a wide range of patients, are better aligned with the device, and/or facilitate extraction of the device.

### BRIEF DESCRIPTION OF THE DRAWINGS

The invention may be more completely understood in consideration of the following detailed description of various embodiments of the invention in connection with the accompanying drawings, in which:

FIG. 1 is a perspective view of a system according to an embodiment of the present invention.

FIG. 2 is a detail view of the tip portion depicted in FIG. 1.

FIG. 3 is a sectional view taken along the line A-A in FIG. 2.

FIG. 4 is a perspective view of an anchor in a retracted delivery position, according to an embodiment of the present invention.

FIG. 5 is a perspective view of an anchor in a deployed position, according to an embodiment of the present invention.

FIG. 6 is a sectional view of a portion of the system of FIG. 1.

FIG. 7 is a detail view of a portion of FIG. 6, depicting an anchor.

FIG. 8 is a sectional detail view of the implant tool of FIG. 1.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE FIGURES

In the following detailed description of the various embodiments of the present invention, numerous specific details are set forth in order to provide a thorough understanding of various embodiments of the present invention. However, one skilled in the art will recognize that the present invention may be practiced without these specific details. In other instances, well-known methods, procedures, and components have not been described in detail so as to not unnecessarily obscure aspects of the various embodiments of the present invention.

The various embodiments of the present invention describes intravascular electrophysiological systems that may be used for a variety of functions to treat cardiac arrhythmias with electrical stimulation. These functions include defibrillation, pacing, and/or cardioversion. In general, the elements of an intravascular implantable device 100 (referred to herein as “IID” or “device”) for electrophysiological therapy include at least one elongate device body 104 and typically, but optionally, at least one lead 108 coupled to the body. While the various embodiments of the present invention is directed to anchoring and retention of the device body of IID 100, it will be understood that, in some embodiments, the one or more leads may also be anchored or retained in the vasculature or within the heart. Alternatively, IID 100 may have no leads, such as for an embodiment of an intravascular implantable drug/gene therapy device, or the one or more leads may not be anchored or retained in the vasculature or within the heart.

In one embodiment, IID 100 includes components, known in the art to be necessary to carry out the system functions of an implantable electrophysiology device. For example, IID 100 may include one or more pulse generators, including associated batteries, capacitors, microprocessors, communication circuitry and circuitry for generating electrophysiological pulses for defibrillation, cardioversion and/or pacing. IID 100 may also include detection circuitry for detecting arrhythmias or other abnormal activity of the heart. The specific components to be provided in IID 100 will depend upon the application for the device, and specifically whether IID 100 is intended to perform defibrillation, cardioversion, and/or pacing along with sensing functions.

IID 100 can be proportioned to be passed into the vasculature and to be anchored within the vasculature of the patient with minimal obstruction to blood flow. Suitable sites for introduction of IID 100 into the body can include, but are not limited to, the venous system using access through the right or left femoral vein or the right or left subclavian vein.

In one embodiment, IID 100 can have a streamlined maximum cross sectional diameter which can be in the range of 3-15 mm or less, with a maximum cross-sectional diameter of 3-8 mm or less in one embodiment. The cross-sectional area of IID 100 in the transverse direction (i.e. transecting the longitudinal axis) can preferably be as small as possible while still accommodating the required components. This area can be in the range of approximately 79 mm̂2 or less, in the range of approximately 40 mm̂2 or less, or between 12.5-40 mm̂2, depending upon the embodiment and/or application.

Additional information pertaining generally to the construction, arrangement and function of an IID suitable for use with the various embodiments of the present invention can be found in U.S. Pat. Nos. 7,082,336, 7,363,082 and 7,529,589, and in U.S. Published Patent Application Nos. 2005/0228471, 2007/0265673, 2008/0147168 and 2008/0167702, the disclosures of which are hereby incorporated by reference.

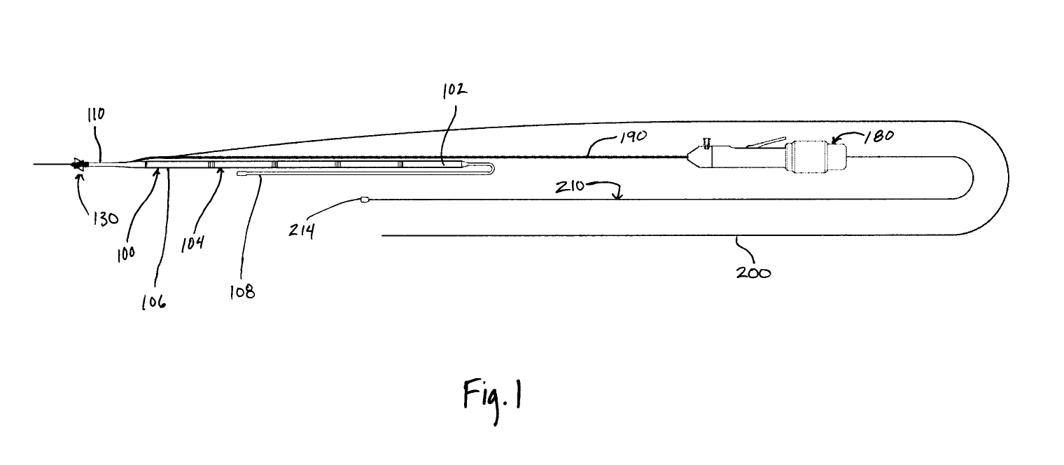

Referring now to FIG. 1, an intravascular implantable device 100 (HD) is provided having a proximal end portion 102, a body portion 104, and a distal end portion 106. Coupled to, integrated with, or otherwise combined with distal end portion 106 is a tip portion 110 (or device tip) extending distally from distal end portion 106 of IID 100. Coupled to, integrated with, or otherwise combined with tip portion 110 is anchor mechanism 130, including an anchor 132 configured to be controllably deployed so as to generally maintain tip portion 110 and/or distal end portion 106 of IID 100 secured within a blood vessel.

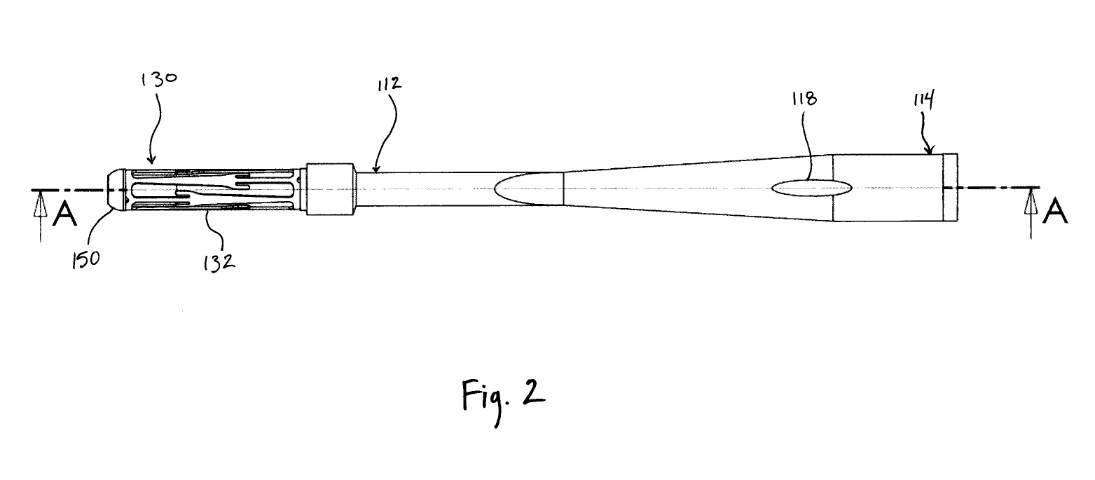

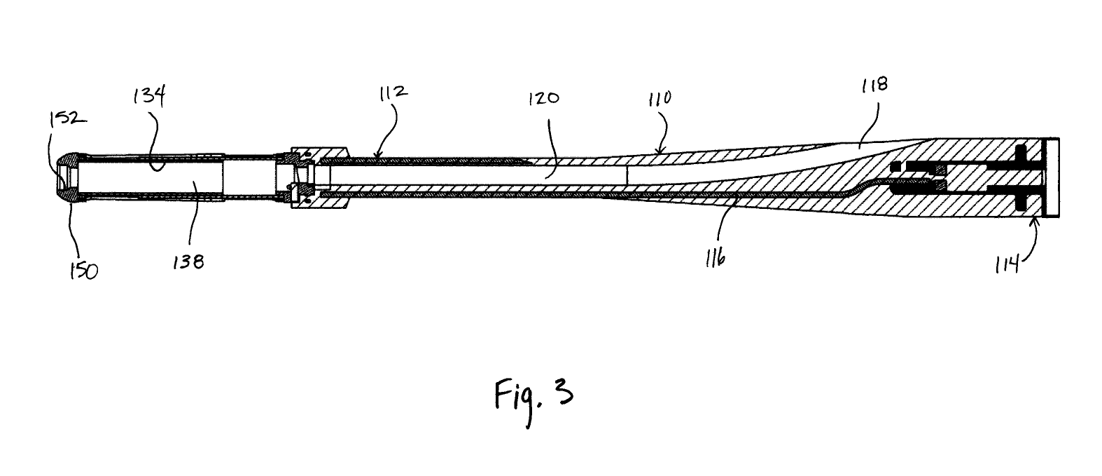

Referring additionally to FIGS. 2 and 3, tip portion 110 includes a distal portion 112 and a proximal portion 114, and is sufficiently flexible to allow bending during implantation yet is preferably more rigid than, for example, a conventional cardiac lead body. Various flexible bio-compatible materials such as Elasteon or silicone, or other materials known in the art may be utilized. Tip portion 110 may include an internal cable 116 to provide axial tensile strength. The proximal portion 114 of tip portion 110 is configured to be non-releasably coupled with the distal end 106 of IID 100, such as by welding or other methods of reliable fastening during manufacturing. Tip portion 110 includes a side-port lumen 118 in communication with a central lumen 120. Central lumen 120 extends through distal portion 112 of tip portion 110.

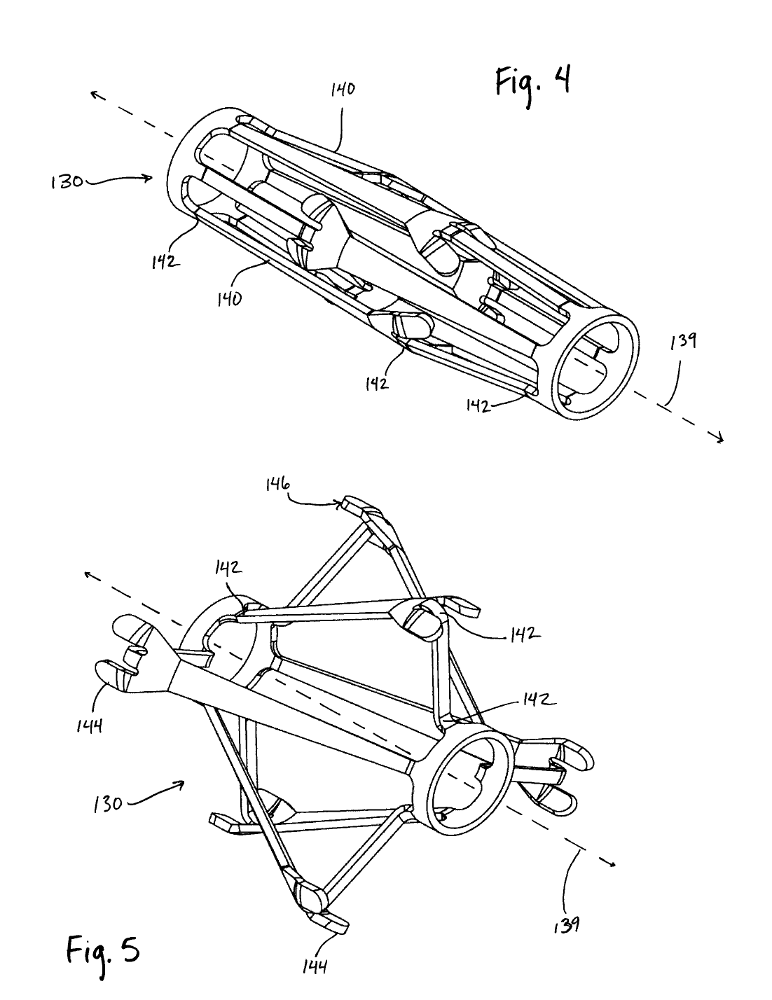

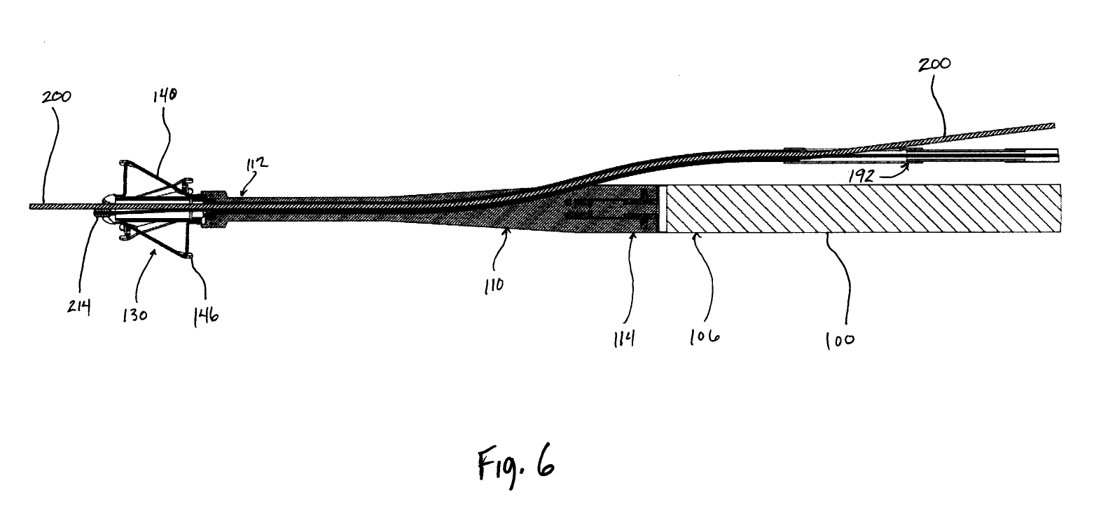

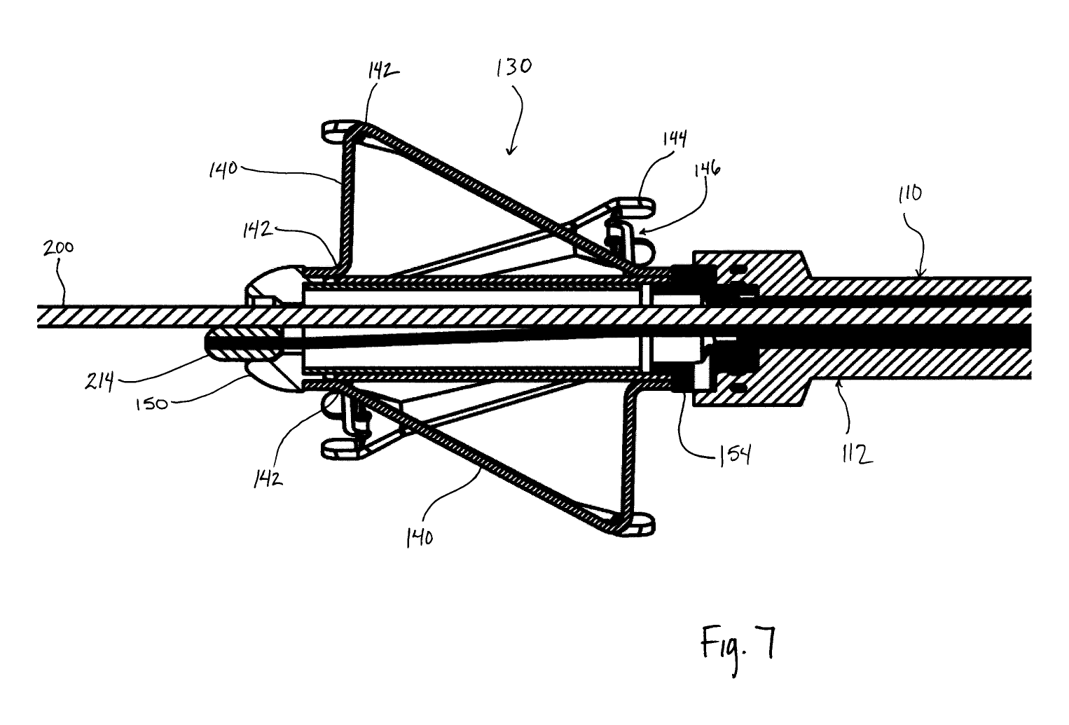

As depicted in FIG. 4, anchor mechanism 130 is generally cylindrical, and coupled to or integrated with tip portion 110. In one embodiment, anchor mechanism 130 is disposed on the distal end 112 of the tip portion 110. Anchor mechanism 130 generally comprises an anchor or retention device 132, a telescoping arrangement 134 with a plurality of segments 136 and defining a central lumen 138 therein, a longitudinal axis 139, and a plurality of leg members 140. The central lumen 138 within anchor 132 is configured to be aligned and in communication with central lumen 120 of tip portion 110. Anchor mechanism 130 is provided in a retracted delivery position, as in FIG. 4, and is configured to be moveable to an extended deployed position, as in FIG. 5, by being compressed in an axial direction along longitudinal axis 139, the axial movement of which causes leg members 140 to be deployed in a direction at a pivot angle relative to the longitudinal axis that is less than 90 degrees.

Operation of anchor mechanism 130 is a departure from prior stent-based anchors, in that a traditional stent expands radially outward, increasing diameter while maintaining a generally constant longitudinal length. The many individual struts of some stents are configured to change orientation from the compressed delivery state of the stent, to the expanded deployed state. This strut re-orientation occurs in a circumferential cylinder of the struts with respect to a longitudinal axis. In contrast, leg members 140 of the present invention deploy in a plane that would be defined transverse to such a circumferential cylinder and are deployed at least one pivot angle relative to the longitudinal axis being less than 90 degrees.

Telescoping arrangement 134 can act as a stop to prevent over-compression and provide stability to anchor body 132 during deployment of the anchor. Leg members 140 may be jointed or hinged at pivot points 142 to facilitate operation of the anchor mechanism, and may operate similar to a linkage. In such an embodiment, points 142 comprise a pin or hinge. In another embodiment, leg members 140 may deform at pre-selected points 142, which have been notched or annealed to promote deformation at that point. In another embodiment, anchor mechanism 130 is provided without a telescoping arrangement.

Referring generally to the Figures, leg members 140 include three hinge points 142. However, it should be appreciated that leg members 142 may include four hinge points 142, or another number of hinges may be used such that the leg member may comprise a 4-bar linkage. Additionally, leg members 142 may be hinged only on each end of anchor body 132, such that axial compression of anchor mechanism 130 causes leg members 140 to bow radially in a curved fashion. Leg members 140 may feature prongs, barbs, or other engagement features 144 on leg tip 146 to enhance engagement with the vessel wall. In one embodiment, leg members 140 may be oriented alternately, such as depicted in FIGS. 4 and 5. In another embodiment, leg members 140 may be configured so as to be oriented in the same direction. Leg members 140 are constructed of a sturdy yet deformable material such as stainless steel. The profile and arrangement of leg members 140 depicted in the Figures should be considered exemplary, not limiting, as modifications may be made while remaining within the scope and spirit of the present invention. An antithrombotic coating is preferably applied to all portions of anchor mechanism 130, including leg members 140.

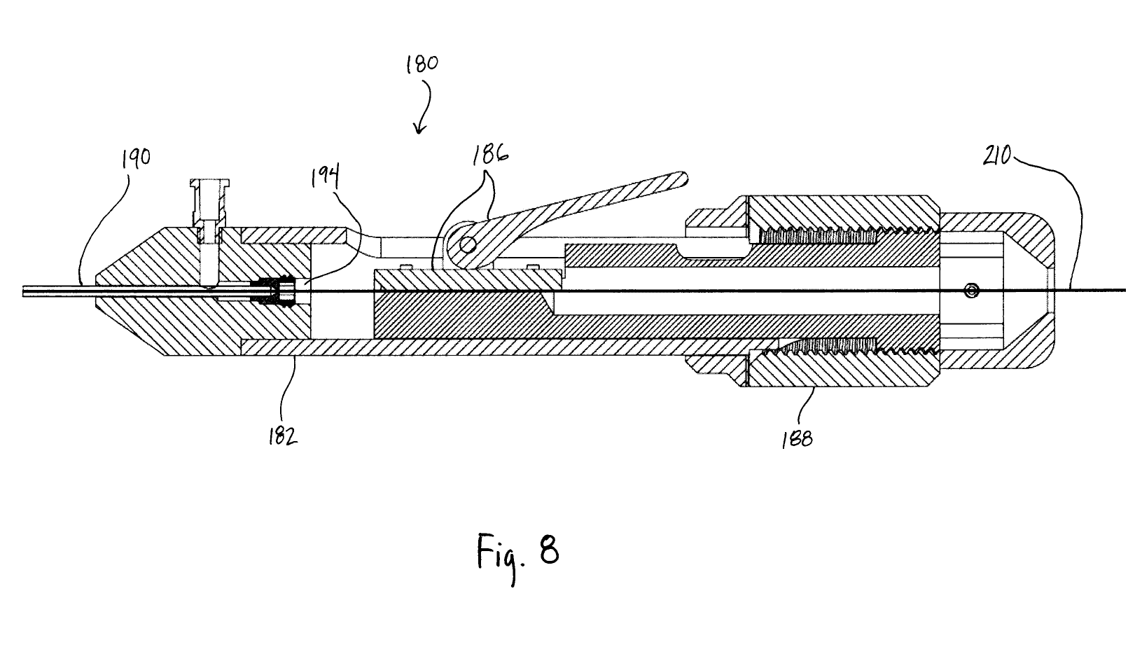

Anchor mechanism 130 further includes a distal cap 150 secured to anchor 132, as depicted in FIG. 7. Distal cap 150 includes a smooth outer profile, and an inner seat 152 in communication with lumen 138. A connection coupler 154 on the proximal end of anchor mechanism 130 serves as the connection to tip portion 110 of IID 100.

In one embodiment, the present invention also comprises implant tools for implanting IID 100 with integrated anchor mechanism 130, as depicted in FIG. 1. Such tools include an anchor delivery catheter (ADC) 180, a guidewire 200, and a pullwire 210. The ADC 180 generally includes a handle portion 182 coupled to a catheter portion 190, with a longitudinal lumen 194 extending throughout. Handle portion 182 may include a clamp 186, and a means 188 for controllably translating a wire within the longitudinal lumen. Means 188 may comprise a rotational mechanism, as depicted in FIG. 8, such that rotation of one portion with respect to the other retracts or extends pullwire 210. The movement of the pullwire by the mechanism is preferably a fine adjustment, allowing precise control of the extension of the anchor mechanism. Catheter portion 190 may comprise a coiled portion 192 on its distal end, and catheter 190 may be steerable and/or torqueable. The coiled portion 192 provides column strength for traction, yet allows a degree of flexibility. Pullwire 210 includes a body 212 with a generally uniform diameter along its length, terminating in a flared or enlarged bulb tip portion 214.

Referring now to implanting IID 100, embodiments of the present invention may comprise methods for implanting device 100 having an integrated anchor mechanism 130. One method comprises obtaining vascular access through a femoral incision, and maintaining access with an introducer sheath having a hemostasis valve. The guidewire 200 is first introduced into the vasculature and directed to a desired location. In one embodiment, the desired location may be superior to the heart such as in the subclavian vein or brachiocephalic vein.

As mentioned above, intravascular implantable device 100 is provided with a distally-located tip portion 110 having an integrated anchor mechanism 130. The integrated anchor 130 and tip portion 110 include a lumen extending throughout, extending from the distal-most end of anchor 132 to the side-port lumen 118 proximate the coupling of the tip portion 110 to the distal portion 106 of IID 100.

IID 100 is loaded with pullwire 210 having a bulb tip 214, such that bulb tip 214 is distal or “in front” of anchor 132, with the pullwire body 212 extending through the lumen 138 in the anchor and the lumen 120 in the tip 110, emerging from the side-port lumen 118 in the tip. IID 100 is loaded onto guidewire 200, distal end first, and advanced into the vasculature. IID 100 is advanced along the guidewire 200 through the vasculature, with guidewire 200 passing through the same lumens in the anchor and IID tip as the pullwire. Anchor delivery catheter (ADC) 180 is introduced onto guidewire 200 and pullwire 210 after IID 100 has been loaded. The ADC 180 includes coiled distal portion 192 adapted to be snugged up against the side-port 118 of IID 100 tip portion 110. The ADC 180 is configured to receive both guidewire 200 and pullwire 210, although the catheter body 190 may include an exit portion for the guidewire. The pullwire 210 passes through the ADC body and into the ADC handle portion 182.

IID 100, pullwire 210 and catheter ADC catheter portion 190 are advanced to the desired location. To activate anchor mechanism 130 to deploy anchor 132, the guidewire 200, pullwire 210 and ADC 180 are utilized. The tolerances between the guidewire, pullwire bulb 214 and the anchor cap 150 are configured such that when guidewire 200 and pullwire 210 both occupy lumen 138 in anchor 132, the pullwire bulb 214 cannot be retracted past cap 150 to enter the lumen, such that pullwire 210 and guidewire 200 are essentially wedged together. The pullwire can then be retracted until bulb tip 214 contacts anchor 132. The clamp 186 on ADC handle 182 is then used to grasp the pullwire, coupling pullwire 210 and ADC 180 with respect to each other. The physician can then operate a mechanism on the ADC handle to progressively retract the pullwire with respect to the ADC, which will begin to compress the anchor mechanism, shortening the anchor in an axial direction while extending the leg members in the radial direction toward the vessel wall.

The degree to which anchor 132 is axially compressed (and therefore the degree to which leg members 140 are extended) is entirely controllable by the physician with ADC 180. Fluoroscopy may be utilized to provide visualization throughout the implant, including deployment of anchor 132. The ADC mechanism may comprise a rotational mechanism (as pictured) such that rotation of one portion with respect to the other retracts or extends the pullwire which is clamped to the ADC. The movement of the pullwire by the mechanism is preferably a fine adjustment, allowing precise control of the anchor mechanism.

In one embodiment, when anchor 132 is satisfactorily engaged with the vessel wall, anchor mechanism 130 and at least tip portion 110 are positioned generally in the center of the blood vessel, in a coaxial fashion. In another embodiment, leg members 140 may be configured with differing hinge points 142 such that the leg members deploy by differing amounts, resulting in anchor mechanism 130 and tip portion 110 being offset from the center of the vessel, while still remaining spaced apart from the inner vessel wall.

After successful anchoring of IID 100, removal of the implant tools begins by releasing clamp 186 on ADC 180. Pullwire 210 is pushed further into the vasculature so as to dislodge pullwire bulb tip 214 from guidewire 200 and anchor cap 150, and guidewire 200 is then removed from anchor 132 and tip portion lumen 120. Guidewire 200 may be fully retracted from the patient at that time if desired. The pullwire 210 and ADC 180 can then be removed from the patient, and additional portions of IID 100 implant procedure can be performed if needed, such as delivery and fixation of one or more leads.

In an alternate embodiment, the compression of the anchor mechanism may be accomplished with a screw mechanism instead of the axial compression deployment of the above-described embodiment. A rotatable screw mechanism is provided within the anchor body, and may be combined with, or separate from the telescoping arrangement. The rotatable screw mechanism may include a screwhead accessible through central lumen 120. In place of pullwire 210, a catheter having a screwdriver-type tip may be provided or integrated into the ADC tool, so as to operate the screw mechanism.

In a further alternate embodiment, the anchor mechanism may include incremental deployment control through the use of a ratcheting mechanism in the anchor. This may be desirable especially in an embodiment wherein the leg members of the anchor are comprised of shape-memory alloy or other springy material. A ratcheting mechanism may be employed to prevent the anchor from returning to the retracted position. In one embodiment, the ratcheting mechanism may be provided as part of the telescoping arrangement, such that one telescoping member includes a plurality of grooves, and another telescoping member includes a tab or ratchet. Operation of such a ratcheting mechanism is similar to that of a cable tie. A release may be provided as part of the ratcheting mechanism.

In another alternate embodiment, the anchor mechanism may be coupled to a powered drive mechanism, such as a stepper motor, screw drive, belt or band drive or a cable drive, and an associated control unit for purposes of automatically controlling the deployment and/or retraction of the anchor mechanism.

In the event that extraction of IID 100 according to the various embodiments of the present invention is required, a cutting tool may be introduced into the vasculature and advanced proximate the anchor to cut the tip portion from the anchor. The anchoring arrangement of the various embodiments of the present invention places the anchor and distal tip of the device generally within the center of the vessel, or at least away from the vessel wall, allowing easy access for a cutting procedure without the risk of damage to the wall.

In one embodiment, instructions for implanting IID 100 in accordance with the various embodiments described herein in the form of printed or electronically, optically or magnetically stored information to be displayed, for example, are provided as part of a kit or assemblage of items prior to surgical implantation of the IID 100. In another embodiment, instructions for implanting the IID 100 in accordance with the various embodiments described herein are provided, for example, by a manufacturer or supplier of IID 100 separately from providing the IID 100, such as by way of information that is accessible using the Internet or by way of seminars, lectures, training sessions or the like.

Various embodiments of systems, devices and methods have been described herein. These embodiments are given only by way of example and are not intended to limit the scope of the present invention. It should be appreciated, moreover, that the various features of the embodiments that have been described may be combined in various ways to produce numerous additional embodiments. Moreover, while various materials, dimensions, shapes, implantation locations, etc. have been described for use with disclosed embodiments, others besides those disclosed may be utilized without exceeding the scope of the invention.

It should be pointed out that many of the retention devices and methods, implantation methods and other features are equally suitable for use with other forms of intravascular implants. Such implants might include, for example, implantable neurostimulators, artificial pancreas implants, diagnostic implants with sensors that gather data such as properties of the patient's blood (e.g. blood glucose level) and/or devices that deliver drugs or other therapies into the blood from within a blood vessel.

Persons of ordinary skill in the relevant arts will recognize that the invention may comprise fewer features than illustrated in any individual embodiment described above. The embodiments described herein are not meant to be an exhaustive presentation of the ways in which the various features of the invention may be combined. Accordingly, the embodiments are not mutually exclusive combinations of features; rather, the invention may comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims for the various embodiments of the present invention, it is expressly intended that the provisions of Section 112, sixth paragraph of 35 U.S.C. are not to be invoked unless the specific terms “means for” or “step for” are recited in a claim.

1. A method of implanting an intravascular implantable device into a vasculature of a patient, comprising:
creating an access to a vasculature of a patient;
advancing the intravascular implantable device until it is fully within the vasculature of the patient, the intravascular implantable device including an integrated anchor and an elongated enclosure containing a battery and electronic components; and
deploying the anchor by compressing the anchor in an axial direction, so as to secure the intravascular implantable device within the vasculature of the patient.

2. The method of claim 1, further comprising:
introducing a guidewire into the access and advancing the guidewire through the vasculature toward a heart of the patient; and
advancing the intravascular implantable device over the guidewire until it is fully within the vasculature of the patient.

3. The method of claim 1, further comprising:
providing a pullwire having a bulb tip on a distal end, such that the bulb tip is positioned distally of the distal end of the anchor;
providing an anchor delivery catheter, wherein a proximal portion of the pullwire is received by the anchor delivery catheter;
using the anchor delivery catheter to retract the pullwire with respect to the anchor delivery catheter such that the anchor is compressed in an axial direction, thereby deploying the anchor to secure the intravascular implantable device within the vasculature of the patient.

4. The method of claim 1, further comprising:
deploying the anchor by compressing the anchor in an axial direction so as to anchor the intravascular implantable device such that the distal tip portion of the device is positioned generally coaxially within the vasculature.

5. An intravascular implantable device, comprising:
an elongated enclosure containing a battery and electronic components; and
an integrated anchor mechanism operably coupled to the elongated enclosure, the anchor mechanism configured to be deployable in response to compression of the anchor mechanism in an axial direction.

6. The intravascular implantable device of claim 5, wherein the integrated anchor mechanism comprises:
a proximal portion configured to be coupled to the implantable intravascular device;
a distal portion;
a longitudinal axis between the proximal portion and the distal portion; and
a plurality of leg members, each leg member including a plurality of joints, each leg member configured to bend transversely outward with respect to the longitudinal axis in response to the retention device being compressed along the longitudinal axis.

7. The intravascular implantable device of claim 5, wherein the intravascular implantable device is flexible about a longitudinal axis at one or more points

8. The intravascular implantable device of claim 5, wherein the anchor mechanism is coupled to a distal end of the elongated enclosure.

9. The retention device of claim 5, further comprising a plurality of telescoping segments extending between the proximal portion and the distal portion.

10. The retention device of claim 6, wherein the plurality of joints comprise hinges.

11. The retention device of claim 6, wherein the plurality of joints are deformable portions of a leg member.

12. A system, comprising:
an intravascular implantable device;
an anchor having a longitudinal axis and a plurality of leg members, each leg member including a plurality of joints;
a pullwire configured to releasably couple to the anchor; and
an anchor delivery catheter, wherein a proximal portion of the pullwire is received by the anchor delivery catheter and the anchor delivery catheter is configured to retract the pullwire with respect to the anchor delivery catheter such that the anchor is compressed along the longitudinal axis and deployed so as to secure the intravascular implantable device within the vasculature of a patient.

13. The system of claim 12, wherein the anchor further comprises a plurality of leg members, each leg member including a plurality of joints, wherein each leg member is configured to bend at the plurality of joints transversely outward with respect to the longitudinal axis in response to the retention device being compressed along the longitudinal axis.

14. A method, comprising:
providing an intravascular implantable device having an integrated anchor mechanism, the intravascular implantable device including an integrated anchor and an elongated enclosure containing a battery and electronic components; and
providing instructions for implanting the intravascular implantable device into a vasculature of a patient, including:
creating an access to a vasculature of a patient;
advancing the intravascular implantable device until it is fully within the vasculature of the patient; and
deploying the anchor by compressing the anchor in an axial direction so as to secure the intravascular implantable device within the vasculature of the patient.
